(19) United States
(12) Patent Application Publication
(10) Pub. No.: US 2012/0178971 A1
(43) Pub. Date: Jul. 12, 2012

(54) Process for Isolation of a Novel Compound 5,8A-DI-1-Propyl 1-Octahydronapthalen-1-(2H) One,Trachyspermum AMI (Ajowan Caraway) Seeds

(75) Inventors: Asad Ullah KHAN, Aligarh (IN); Rosina KHAN, Aligarh (IN)

(73) Assignees: ALIGARH MUSLIM UNIVERSITY, Aligarh (IN); DEPARTMENT OF BIOTECHNOLOGY, New Delhi (IN)

(21) Appl. No.: 13/343,883

(22) Filed: Jan. 5, 2012

(30) Foreign Application Priority Data
Jan. 6, 2011 (IN) 24/DEL/2011

(51) Int. Cl. C07C 45/81; C07C 49/447

(52) U.S. Cl. 568/366; 568/374

(57) ABSTRACT

A novel compound 5,8a-di-1-propyl 1-octahydronapthalen-1-(2H) One, and a process for isolating the compound and provides the process for isolation of the compound 5,8a-di-1-propyl 1-octahydronapthalen-1-(2H) One from Trachyspermum ammi (Ajowan caraway) seeds involving washing the seeds, drying the same and crushing it to form a powder; subjecting the powder to solvent extraction; isolating the active compound by chromatography; cooling the fractions; and subjecting the fractions to the step of crystallization in pure methanol.

### FIELD OF INVENTION

This invention relates to novel compound “5,8a-di-1-propyl 1-octahydronapthalen-1-(2H) one, from Trachyspermum (Ajowan caraway) seeds used for the prevention and treatment of Dental caries”

### BACKGROUND OF THE INVENTION

This is first isolated compound from Ajowan caraway seeds. Dental caries is most prevalent disease, next only to the common cold. It causes an irreversible damage to the grinding machinery involved in the intake of the food and hence, causes great discomfort. The changes in homeostasis of oral cavity with an overgrowth of Streptococcus mutans is recognized as the primary cause of the disease. Hence, most of the targets are now aimed at either elimination of this bacteria or suppression of its virulence. S Mutans strongly adhere and release acids by the fermentation of carbohydrates that leads to the demineralization of the tooth. This attachment is mediated mostly by the interaction of the surface proteins and extra cellular polysaccharide of bacteria. The ambiguity is basic treatment for dental caries like the use of fluoride and antibiotics vitalizes the deployment of herbal medicines. We therefore isolated and characterized a compound from Ajowan seeds to treat Dental caries with no side effects.

### OBJECTS OF THE INVENTION

An object of this invention is to propose a process for isolation of a novel compound 5,ba-di-1propy1-octachydronapthalen-1-(2H)one from Trachyspermum (Ajowan caraway) seeds

Another object of this invention is to propose a compound which is used for the prevention and treatment of dental caries.

Further object of this invention is to propose a novel compound which possesses anti-biofilm & anti adherence properties.

Still further object of this invention is to propose a compound which is anticariogenic activity.

### BRIEF DESCRIPTION OF THE INVENTION

According to this invention there is provided a novel compound 5,8a-di-1-propyl 1-octahydronapthalen-1-(2H) One.

In accordance with this invention there is provided a process for isolation of a novel compound 5,8a-di-1-propyl 1-octahydronapthalen-1-(2H) One, from Trachyspermum (Ajowan caraway) seeds for the prevention and treatment of Dental caries comprising

Washing the seeds, drying the same and crushing it to form a powder,

Subjecting the powder to the step of solvent extraction

Isolating the active compound by chromatography

Cooling the fractions

And subjecting the fractions to the step of crystallization in pure methanol.

### BRIEF DESCRIPTION OF THE ACCOMPANYING DRAWINGS

FIG. 1: A reaction Scheme.

FIG. 2: Inhibitory effect of the compound on the extent of sucrose dependent cell adherence of Streptococcus mutans. The % cell adherence is the relative amount (%) of adherent cells at different concentrations of the compound as compared to the cells attached in the absence of any compound. Each value is an average of triplicate assays and each bar indicates ±SD (n=3).

FIG. 3: Inhibitory effect of the compound on the biofilm formed by Streptococcus mutans. The % biofilm formed is the relative (%) of biofilm formed at different concentrations of the compound as compared to that in the absence of any compound. Each value is an average of triplicate assays and each bar indicates ±SD (n=3)

FIG. 4: CLSM images of Streptococccus mutans biofilm formed in the presence or absence of the compound after 24 h incubation. The results are expressed as the means ±standard deviations of triplicate assays.

FIG. 5: Inhibitory effect of compound on the hyrdophobicity of Streptococcus mutans where all the concentrations were taken in triplicates.

FIG. 6: Inhibitory effects of the compound on insoluble glucan synthesis by crude GTF of Streptococcus mutans. The % insoluble glucan synthesis is the relative amount (%) ±SD (n=3)

### DETAILED DESCRIPTION

This invention relates to a process for the isolation of 5,8a-di-1propy 1-octachydronapthalen-1-(2H)-One.

The seeds were washed, dried under shade and crushed to make powder. The powdered seeds were exhaustively extracted with solvents of increasing polarity.

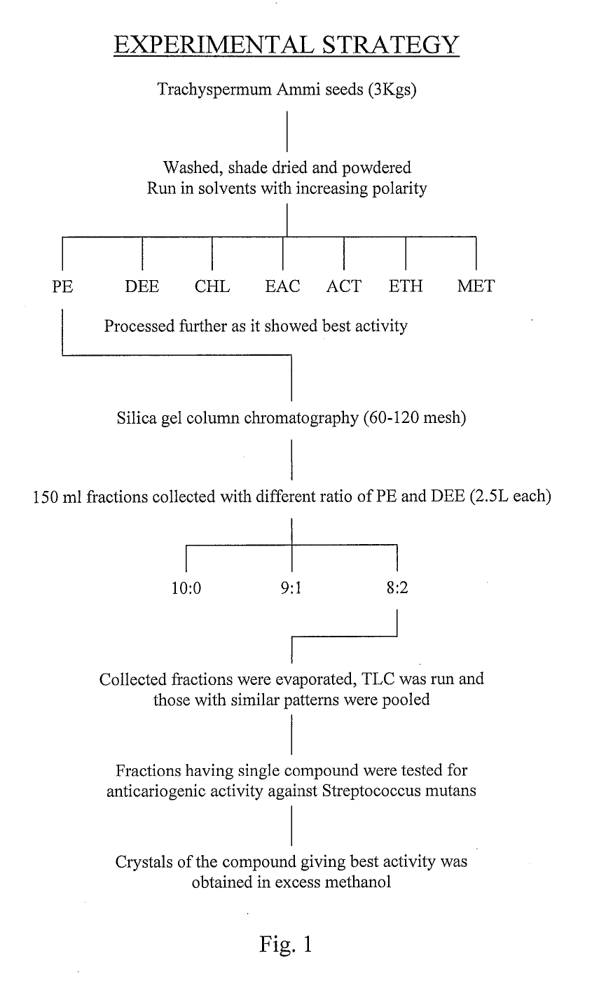

The petroleum ether extract was concentrated under reduced pressure and chromatographed over silica, gel (60-80 mesh) column (110×4cm) for the isolation of the active compound. Fractions of 150 ml were collected and evaporated. Different ratios of petroleum either and diethyl ether were run. The active compound was found in fractions 67 and 68 (petroleum ether: diethyl ether: 8:2), which showed similar pattern on the TLC plates and gave similar FTIR spectra. On crystallization with excess methanol, the active compound, with the help of different spectroscopic techniques was identified as (4aS, 5R, 8aS) 5,8a-di-1-propyl-octahyrdronapthalen-1-(2H)-one. The reaction scheme is shown in FIG. 1.

The purified compound was obtained as white amorphous solid. Its melting point was measured as 82° C. and was uncorrected. The. FTIR spectrum band v (cm⁻¹) at 2920, 2840, 1700, 1440, 1400 1310, 880 and 600 (Figure S1). ¹H NMR spectrum displayed signals at δ0.89(t,J=7.2 H₃-3′ and H₃-3″, δ1.26 (brs, methylene protons), 1.64 (m, J=7.6 H-5 and H-8b) and 2.36 (t, J=7.6 H₂-2) (Figure S2(a), (b)). The ¹³ C NMR gave signals at δc 210.16 (C-1), 40.25 (C-2), 22.69 (C-3), 24.75 (C-4) 35.36(C-5) (C-5), 31.93(C-6), 21.70 (C-7), 33.65(C-8), 52.60(C-8a), 46.45(C-8b), 35.09(C-1′), 19.26 (C-2′ and C-2″), 14.12 (C-3′ and C-3″) and 35.43 (C-1″0)(Figure S3). The positive ion FAB MS spectrum showed peaks at m/z 236, 193,181, 166, 152, 150, 138, 98, 84, 70, 55, 43 and 42 (Figure S4). The base peak was observed at m/z 55. The compound was identified as 5,8a-di-1-propyl-octahydronaptialen-1-(2H)-one. The structure is shown in figure

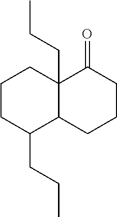

5,8a-di-1 propyl-octahydronapthalen-1-(2H)-one

### Determination of MIC And MBC

The MIC of the compound against Streptococcus mutans was found to be 156.25 μg/ml.

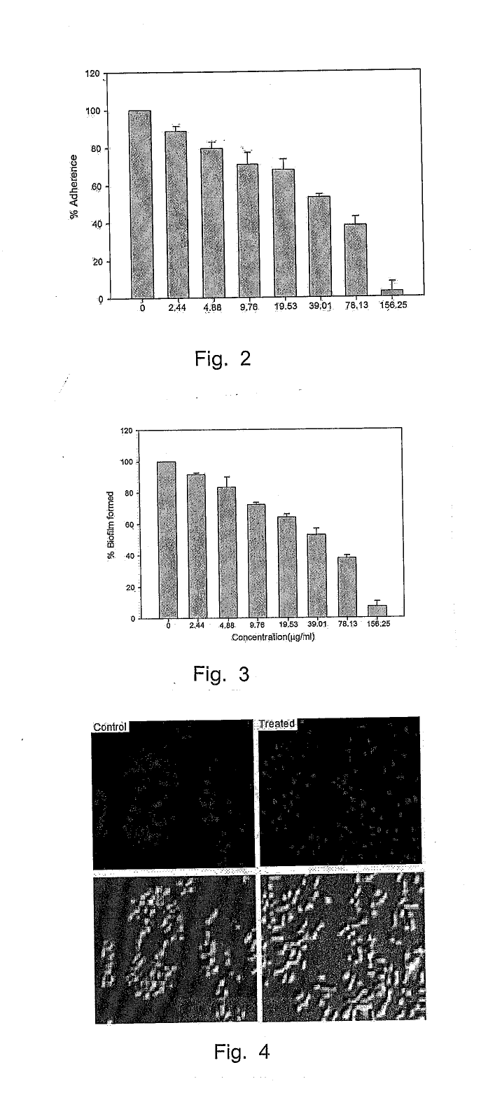

The effect of the adherence of Streptococcus mutans by different concentrations of the compound are given in Figure. The reduction in the adherence was found to be in a dose dependent manner. The least concentration to give at least 50% inhibition was 39.06 μg/ml. Even though the MIC is not very effective, that is, 156.25 μg/ml, the adherence properties were effected at a concentration as low as 9.77 μg/ml with 30% reduction as shown in FIG. 2.

### Effect On Streptococcus Mutans Biofilm Formation

The biofilm formation was assessed using increasing concentration of the compound. At 78.13 μg/ml., the compound inhibited the biofilm formation of Streptococcus mutans. As observed in the adherence, the inhibitory effect was also found to be in a dose dependent manner. As expected, the compound completely inhibited the biofilm formation at 156.25 μg/ml (MIC) due to its antimicrobial effect as shown in FIG. 3.

### Confocal Microscopy

The biofilm of Streptococcus mutans was analyzed under the confocal laser scanning microscope to observe the changes in its morphology as shown in FIG. 4. In the absence of the compound, the cells showed clumps and aggregate that was absent when treated with sub inhibitory concentration of the compound. The cells in the treated sample were more spread out and dispersed. Each panel of the image is a representative view of 141.145 μm by 141.145 μm along the xy axis. The xy analysis provides the biofilm surface coverage, while the z-section analyses establish the thickness of the biofilm. The control cells have biofilms that are clumped and less spread along the xy lane, show a thickness of 19.74±0.5 μm. In the presence of 78.13 μ/ml of the compound, the cells show a complete absence of clumped cells and thickness is 12.8±0.7 pm. The cells wee individually scattered over the surface rather than in any arrangement.

### Inhibition of Acid Production

The pH of the culture medium was recorded after 24 h of treatment to determine the effect of this compound on acid production. The reduction in pH drop at various concentrations of the compound is reported in table. At a concentration of 78.13 μg/ml, there is maximum reduction in acid production as is evident by an increase in pH from 4.7 (control) to 7.5. This suggests that 78.13 μg/ml compound effectively inhibits acids production in Streptococcus mutans.

| CONCENTRATION | pH ± SD | pH ± SD |
|---|---|---|
| (μg/ml) | (onset) | (after 24 h) |
| 0 | 7.44 ± 1.09 | 4.71 ± 3.41 |
| 2.44 | 7.28 ± 0.09 | 4.77 ± 1.69 |
| 4.88 | 7.24 ± 1.33 | 4.84 ± 1.20 |
| 9.76 | 7.33 ± 1.11 | 5.49 ± 1.05 |
| 19.53 | 7.38 ± 1.91 | 5.62 ± 1.96 |
| 39.01 | 7.43 ± 1.13 | 5.68 ± 6.78 |
| 78.13 | 7.43 ± 0.15 | 6.48 ± 5.97 |
| 156.25 | 7.38 ± 1.29 | 7.55 ± 2.65 |

### Effect of the Compound On Hydrophobocity of Streptococcus Mutans

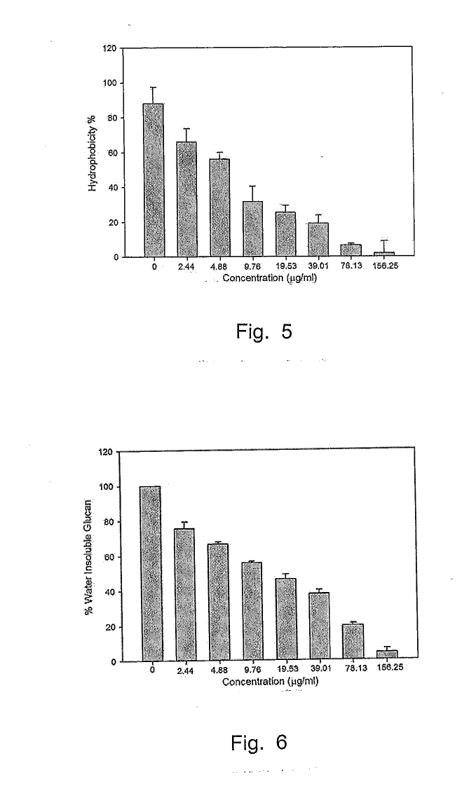

It was shown that the cell surface hydrophobocity of S. mutans is affected following treatment with the compound obtained from Trachyspermum ammi as shown in figure. Exposing the bacteria to 9.76 μg/ml of the compound drastically reduced the cell surface hydrophobicity to more than half when compared to untreated condition as shown in FIG. 5.

### Inhibition of Water Insoluble Glucan Synthesis

The influence of different concentrations was assessed for the synthesis of insoluble glucan. The synthesis was found to be suppressed by the compound in dose dependent manner. There was a 25% reduction in the synthesis of insoluble glucan at a concentration as low as 4.88 μl/ml as shown in FIG. 6.

1. A 5,8a-di-1-propyl 1-octahydronapthalen-1-(2H) One compound comprising a structure as shown below:

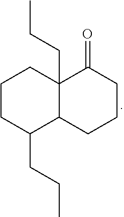

2. (canceled)

3. A process for the isolation of 5,8a-di-1-propyl 1-octahydronapthalen-1-(2H) One from Trachyspermum ammi (Ajowan caraway) comprising:
washing the seeds, drying the same and crushing it to form a powder;
subjecting the powder to solvent extraction;
isolating an active compound by chromatography;
cooling the fractions; and
subjecting the fractions to crystallization in presence of methanol.

4. The process according to claim 3, wherein the powder is extracted with solvents of increasing polarity.

5. The process according to claim 3, wherein said solvent is petroleum ether extract concentrated under pressure and chromatographed over a silica gel column.

6. The process according to claim 3, wherein the active compound is isolated by chromatography in a silica gel column.
